(19) United States
(12) Patent Application Publication
(10) Pub. No.: US 2026/0294787 A1
(43) Pub. Date: Oct. 1, 2026

(54) MINERAL OIL-FREE, MECHANICALLY STABLE LIPSTICK

(71) Applicant: BEIERSDORF AG, Hamburg (DE)

(72) Inventors: Sepideh RESHAD, Hamburg (DE); Hendrik KUELPER, Schenefeld (DE)

(21) Appl. No.: 18/881,850

(22) PCT Filed: Jun. 27, 2023
(86) PCT No.: PCT/EP2023/067411

§ 371 Date: Jan. 7, 2025

(30) Foreign Application Priority Data
Jul. 11, 2022 (DE) 10 2022 207 046.1

(51) Int. Cl. A61K 8/92; A61K 8/02; A61K 8/55; A61K 8/67; A61Q 1/06

(52) U.S. Cl. CPC A61K 8/927; A61K 8/0229; A61K 8/553; A61K 8/676; A61K 8/922; A61Q 1/06; A61K 2800/34

(57) ABSTRACT

Lipstick containing a combination of a) beeswax (INCI: Cera alba), b) rapeseed wax (INCI: Hydrogenated Rapeseed Oil) and c) sunflower oil obtained from sunflower hybrids (INCI: Helianthus annuus Hybrid Oil).

The present invention relates to a lipstick comprising a combination of
beeswax (INCI: Cera alba), rapeseed wax (INCI: Hydrogenated Rapeseed Oil) and sunflower oil obtained from sunflower hybrids (INCI: Helianthus annuus Hybrid Oil).

The desire to look beautiful and attractive has been rooted in humans for thousands of years. Although ideals of beauty have changed over time, the pursuit of a flawless appearance has always been aimed for by humans, since a pleasant appearance increases their self-esteem and attractiveness to their fellow human beings.

The term “decorative cosmetics” comes from the Latin “decoratio”—the emphasizing of beauty. In most cases, colorants are used to emphasize individual parts of the body, especially on the face, and to lessen color non-uniformities.

In addition to face powders and rouge as powdered cosmetics and stick-shaped preparations, creamy preparations such as day creams and cream make-up based on emulsions are used for this purpose.

Lipstick is one of the most used decorative cosmetics. More than 50% of women in Germany use it. In addition to accentuating the color of the lips, it usually also serves for lip care.

Lipsticks generally consist of a wax matrix in which high concentrations of liquid and semi-solid oils and also pigments and fillers are incorporated. They are in solid stick form.

A disadvantage of the prior art is the fact that a relatively large proportion of scrap is produced in the production of lipsticks. This phenomenon occurs particularly often in the case of lipsticks that are produced from natural-based raw materials, i.e. that are free of petroleum jelly, paraffins, mineral oils, silicone oils and silicone waxes.

Lipsticks are usually produced by the mold casting process. This involves the hot, liquid preparation being cast into stick molds and then cooled and hardened. When the hardened stick blanks are subsequently removed from the casting mold and filled into the packaging container, the stick compound frequently breaks and cracks. This production scrap increases the complexity and the costs of lipstick production.

It was therefore the object of the present invention to develop a lipstick (in particular based on natural raw materials) in the production of which the amount of this scrap is reduced.

In order to be able to estimate the risk of breakage and cracks in production already during product development, use can be made in the laboratory of the needle penetration test. In this test, two needles of different thicknesses are pressed into the stick compound with constant force and the penetration depth is determined. Particularly robust sticks are characterized by the fact that there is as little as possible a difference in the penetration depth for the needles of different thicknesses. The method is described in more detail below.

In addition to the stability in relation to mechanical actions during production, it is also important that the cosmetic properties of the lipsticks are retained. In particular, the smoothness and the glidability when applied to the lips should be retained. Hard sticks that are insensitive to mechanical loads in production however have the disadvantage of no longer exhibiting this smoothness and glidability.

Therefore, the object of the present invention was to develop a lipstick which retains its sensory-tactile properties in particular with respect to smoothness and glidability upon application, without resulting in high scrap rates during production.

The objects are surprisingly achieved by

A lipstick comprising a combination of
a) beeswax (INCI: Cera alba),
b) rapeseed wax (INCI: Hydrogenated Rapeseed Oil) and
c) sunflower oil obtained from sunflower hybrids (INCI: Helianthus annuus Hybrid Oil, CAS No. 164250-88-8).

According to the invention, this lipstick is advantageously characterized in that the sunflower oil obtained from sunflower hybrids (INCI: Helianthus annuus Hybrid Oil) has an oleic acid content (C18:1) of 60% to 95%, measured in accordance with IUPAC 2.304. An oleic acid content of 70% to 90%, measured in accordance with IUPAC 2.304, is preferred here according to the invention. In contrast, conventional sunflower oils have an oleic acid content (C18:1) of about 14% to 40%, measured in accordance with IUPAC 2.304.

Advantageous embodiments in the sense of the present invention are also characterized in that the sunflower oil obtained from sunflower hybrids (INCI: Helianthus annuus Hybrid Oil) has an acid value of at most 0.22 mg KOH/g, measured in accordance with IUPAC 2.201 (m). An acid value of at most 0.2 mg KOH/g, measured in accordance with IUPAC 2.201 (m), is preferred here according to the invention.

It is also advantageous according to the invention if that the sunflower oil obtained from sunflower hybrids (INCI: Helianthus annuus Hybrid Oil) has a peroxide value of at most 1.1 meq/kg, measured in accordance with AOCS Cd8b-90 (m). A peroxide value of at most 1.0 meq/kg, measured in accordance with AOCS Cd8b-90 (m), is preferred according to the invention.

Embodiments of the lipstick according to the invention that are advantageous according to the invention are characterized in that the preparation comprises ascorbyl palmitate.

A content of ascorbyl palmitate of 0.01% to 0.05% by weight, based on the total weight of the preparation, is advantageous here according to the invention.

It is also advantageous according to the invention if the preparation of the lipstick comprises lecithin.

A content of lecithin of 0.05% to 0.1% by weight, based on the total weight of the preparation, is advantageous here according to the invention.

Embodiments of the lipstick that are advantageous according to the invention are characterized in that the preparation comprises beeswax (INCI: Cera alba) in a concentration of 10% to 18% by weight, based on the total weight of the preparation. A content of 13% to 15% by weight, based on the total weight of the preparation, is preferred here according to the invention.

It is advantageous according to the invention if the preparation of the lipstick comprises rapeseed wax (INCI: Hydrogenated Rapeseed Oil) in a concentration of 8% to 16% by weight, based on the total weight of the preparation. A content of 11% to 13% by weight, based on the total weight of the preparation, is preferred here according to the invention.

It is also advantageous according to the invention if the preparation of the lipstick comprises sunflower oil obtained from sunflower hybrids (INCI: Helianthus annuus Hybrid Oil) in a concentration of 60% to 80% by weight, based on the total weight of the preparation. A content of 65% to 75% by weight, based on the total weight of the preparation, is preferred here according to the invention.

Embodiments of the present invention that are advantageous according to the invention are further characterized in that the preparation comprises shea butter (Butyrospermum parkii Butter). In such a case, it is preferred according to the invention if the preparation comprises shea butter (Butyrospermum parkii Butter) in a concentration of 1% to 7% by weight, based on the total weight of the preparation.

It is also advantageous according to the invention if the preparation comprises tocopherol. This may be used in a concentration of 0.05% to 0.1% by weight, based on the total weight of the preparation.

Embodiments of the present invention that are advantageous according to the invention are also obtained by the preparation being free of 2-ethylhexyl 2-cyano-3,3-diphenylacrylate (Octocrylene), homomenthyl salicylate (Homosalate), 2-ethylhexyl 4-methoxycinnamate, 3-(4-methylbenzylidene)camphor, 2-hydroxy-4-methoxybenzophenone (Oxybenzone), methyl anthranilate (INCI: Methyl Anthranilate) and sulisobenzone (INCI: Benzophenone-4), polyethylene glycol, polyethylene glycol ethers and polyethylene glycol esters (so-called PEG derivatives), being free of polyacrylates, and being free of parabens, methylisothiazolinone, chloromethylisothiazolinone and DMDM hydantoin.

In particular, it is advantageous according to the invention if the lipstick preparation is free of petroleum jelly, paraffins, mineral oils, silicone oils and silicone waxes.

Lipsticks that are advantageous according to the invention are also characterized in that the difference in the penetration depth in the needle penetration test using the Petrotest PNR 10 electronic gravity penetrometer between a 2.5 g standard needle (order no. 18-0063 Petrotest Instruments) and a 2.5 g tapered needle (order no. 18-0081 Petrotest Instruments) with a penetration time of 5 sec at 20° C. is less than 1.8 mm.

Needle penetration is generally a consistency test of highly viscous deformable substances. Here, a needle-shaped or tapered test body is placed with the aid of a plunger (clamping and guide shank), here 47.5 g (order no. 18-0042 Petrotest Instruments) on the surface of a sample, so that it penetrates into the test material under its own weight, composed of plunger and needle, during a fixed period of time. The penetration depth of the test body is measured in millimeters here and is a measure of the plasticity or consistency.

The test was carried out using the Petrotest PNR 10 electronic gravity penetrometer. For the use for testing lip balms, the test material (the lip balm in the packaging) is positioned on the test surface. The test bodies, in this case two different needles (see above), are first clamped into the plunger (see above), then placed on the surface of the lip balm and finally allowed to fall by a release mechanism. In the fixed period of time of 5 sec, the test body then penetrates into the compound of the lip balm by way of its own weight (plunger+needle); after this time has elapsed, the penetration depth can be read.

Furthermore, the lipstick according to the invention may comprise the ingredients that are typical for such products, for example colorants, aroma substances, UV filters, etc.

It is thus preferred according to the invention if the preparation of the lipstick comprises one or more UV filters selected from the following group of compounds: 2,4,6-tris[anilino-(p-carbo-2′-ethyl-1′-hexyloxy)]-1,3,5-triazine (INCI: Ethylhexyl Triazone), 2-ethylhexyl 2-hydroxybenzoate (INCI: Ethylhexyl Salicylate), 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine) and 4-(tert-butyl)-4′-methoxydibenzoylmethane (INCI: Butyl Methoxydibenzoylmethane). According to the invention, particular preference is given here to the use of a combination of 2,4,6-tris[anilino-(p-carbo-2′-ethyl-1′-hexyloxy)]-1,3,5-triazine (INCI: Ethylhexyl Triazone) and 4-(tert-butyl)-4′-methoxydibenzoylmethane (INCI: Butyl Methoxydibenzoylmethane).

Last but not least, the invention provides the process for producing a lipstick according to one of the preceding claims, characterized in that all constituents are melted while stirring, then are cast into a casting mold at a temperature of approx. 57° C. to 63° C. and finally removed once the casting mold together with the cast compound has cooled at 17° C. to 23° C. for a defined period of time.

### Comparative Experiment

The formulations that follow were produced and the needle penetration was determined in accordance with the above-described method according to the invention.

| | For- | For- | For- |
|---|---|---|---|
| | mulation | mulation | mulation |
| Europe INCI | 1 | 2 | 3 |
| Octyldodecanol | 69.6 | | |
| Helianthus Annuus Seed Oil | | 69.6 | |
| Helianthus Annuus | | | 69.6 |
| Hybrid Oil + Lecithin + | | | |
| Ascorbyl Palmitate | | | |
| Butyrospermum Parkii Butter | 4 | 4 | 4 |
| Cera Alba | 14 | 14 | 14 |
| Tocopherol + Helianthus | 0.1 | 0.1 | 0.1 |
| Annuus Seed Oil | | | |
| Hydrogenated Rapeseed Oil | 12 | 12 | 12 |
| Aroma | 0.3 | 0.3 | 0.3 |
| Total | 100 | 100 | 100 |

### Needle Penetration Test

| | | | | Formulation 3 | |
|---|---|---|---|---|---|
| | | | | (Helianthus Annuus | |
| | | | | Hybrid Oil + | |
| Formulation 1 | | Formulation 2 | | Lecithin + Ascorbyl | |
| (Octyldodecanol) | | (Helianthus Annuus Seed Oil) | | Palmitate) | |
| Standard | | Standard | | Standard | |
| needle | Tapered | needle | Tapered | needle | Tapered |
| [mm] | needle [mm] | [mm] | needle [mm] | [mm] | needle [mm] |
| 25.6 | 11.3 | 12.2 | 8.37 | 9.05 | 8.67 |
| 26.1 | 11.1 | 13 | 8.33 | 9.58 | 8.48 |
| 27.9 | 11.2 | 13.7 | 8.13 | 11 | 8.51 |
| 30 | 11.1 | 11.4 | 8.35 | 11.1 | 8.59 |
| 26.3 | 10.8 | 13.5 | 8.68 | 10.1 | 8.75 |
| 28 | 10.7 | 10 | 8.42 | 11.6 | 9.09 |
| 31.9 | 10.7 | 11 | 8.47 | 8.61 | 8.5 |
| 24.9 | 12.5 | 10.9 | 8.62 | 8.61 | 8.55 |
| 25.7 | 11.6 | 9.9 | 8.49 | 9.85 | 8.96 |
| 25.9 | 11.6 | 9.49 | 8.5 | 8.76 | 9.44 |
| 21.4 | 12.3 | 10.6 | 8.59 | 9.18 | 8.68 |
| 24.7 | 11.9 | 9.42 | 8.56 | 9.17 | 8.65 |
| 20.6 | 11.5 | 10.8 | 8.47 | 11.4 | 8.83 |
| 20.6 | 11.8 | 10.2 | 8.76 | 9.54 | 8.75 |
| 21 | 12.1 | 9.93 | 8.3 | 10.6 | 8.68 |
| 18.8 | 11.6 | 9.33 | 8.23 | 9.36 | 8.74 |
| 18 | 11 | 9.42 | 8.31 | 11.7 | 8.72 |
| 18.9 | 11.7 | 9.84 | 8.23 | 11.7 | 8.65 |
| 17.3 | 11.2 | 8.44 | 8.86 | 11.6 | 8.71 |
| 18.3 | 12 | 8.82 | 8.44 | 11.6 | 8.81 |
| Avg 23.60 | Avg 11.49 | Avg 10.59 | Avg 8.46 | Avg 10.21 | Avg 8.74 |

Standard needle/tapered needle difference:
Formulation 1: 12.11 mm
Formulation 2: 2.13 mm
Formulation 3: 1.47 mm

### EXAMPLES

The examples which follow are intended to illustrate the present invention without limiting it. Unless otherwise indicated, all amounts, proportions and percentages are based on the weight and the total amount or on the total weight of the preparations.

| | For- | For- | For- |
|---|---|---|---|
| | mulation | mulation | mulation |
| Europe INCI | Example 1 | Example 2 | Example 3 |
| Helianthus Annuus | 60 | 72 | 63 |
| Hybrid Oil + Lecithin + | | | |
| Ascorbyl Palmitate | | | |
| Butyrospermum Parkii Butter | 6 | 3 | 5 |
| Cera Alba | 14.5 | 11 | 13 |
| Tocopherol + Helianthus | 0.1 | 0.1 | 0.2 |
| Annuus Seed Oil | | | |
| Hydrogenated Rapeseed Oil | 15 | 9 | 14 |
| Butyl | 2 | | |
| Methoxydibenzoylmethane | | | |
| Ethylhexyl Triazone | 2 | | |
| Aroma | 0.4 | 0.4 | 0.3 |
| Ricinus Communis Seed | | 0.7 | |
| Oil + CI 15850 | | | |
| Ricinus Communis Seed | | 0.3 | |
| Oil + CI 15985 | | | |
| Ricinus Communis Seed Oil | | 3.5 | 4.5 |
| Total | 100 | 100 | 100 |

1.-17. (canceled)

18. A lipstick, wherein the lipstick comprises a combination of
(a) beeswax (INCI: Cera alba),
(b) rapeseed wax (INCI: Hydrogenated Rapeseed Oil), and
(c) sunflower oil obtained from sunflower hybrids (INCI: Helianthus annuus Hybrid Oil).

19. The lipstick of claim 18, wherein (c) has an oleic acid content (C18:1) of 60% to 95%, measured in accordance with IUPAC 2.304.

20. The lipstick of claim 18, wherein (c) has an acid value of at most 0.22 mg KOH/g, measured in accordance with IUPAC 2.201 (m).

21. The lipstick of claim 18, wherein (c) has a peroxide value of at most 1.1 meq/kg, measured in accordance with AOCS Cd8b-90 (m).

22. The lipstick of claim 19, wherein (c) further has an acid value of at most 0.22 mg KOH/g, measured in accordance with IUPAC 2.201 (m) and a peroxide value of at most 1.1 meq/kg, measured in accordance with AOCS Cd8b-90 (m).

23. The lipstick of claim 18, wherein the lipstick further comprises ascorbyl palmitate.

24. The lipstick of claim 18, wherein the lipstick further comprises lecithin.

25. The lipstick of claim 18, wherein the lipstick comprises from 10% to 18% by weight of (a), based on a total weight of the lipstick.

26. The lipstick of claim 18, wherein the lipstick comprises from 8% to 16% by weight of (b), based on a total weight of the lipstick.

27. The lipstick of claim 18, wherein the lipstick comprises from 60% to 80% by weight of (c), based on a total weight of the lipstick.

28. The lipstick of claim 25, wherein the lipstick comprises from 8% to 16% by weight of (b) and from 60% to 80% by weight of (c), based on a total weight of the lipstick.

29. The lipstick of claim 22, wherein the lipstick comprises from 10% to 18% by weight of (a), from 8% to 16% by weight of (b) and from 60% to 80% by weight of (c), based on a total weight of the lipstick.

30. The lipstick of claim 18, wherein the lipstick further comprises shea butter (Butyrospermum parkii Butter).

31. The lipstick of claim 30, wherein the lipstick comprises from 1% to 7% by weight of shea butter, based on a total weight of the lipstick.

32. The lipstick of claim 18, wherein the lipstick further comprises tocopherol.

33. The lipstick of claim 18, wherein the lipstick is free of 2-ethylhexyl 2-cyano-3,3-diphenylacrylate (Octocrylene), homomenthyl salicylate (Homosalate), 2-ethylhexyl 4-methoxycinnamate, 3-(4-methylbenzylidene)camphor, 2-hydroxy-4-methoxybenzophenone (Oxybenzone), methyl anthranilate (INCI: Methyl Anthranilate) sulisobenzone (INCI: Benzophenone-4), polyethylene glycol, polyethylene glycol ethers. polyethylene glycol esters, polyacrylates, parabens, methylisothiazolinone, chloromethylisothiazolinone, and DMDM hydantoin.

34. The lipstick of claim 18, wherein the lipstick is free of petroleum jelly, paraffins, mineral oils, silicone oils, and silicone waxes.

35. The lipstick of claim 18, wherein a difference in a penetration depth in a needle penetration test between a standard needle and a tapered needle with a penetration time of 5 sec at 20° C. is less than 1.8 mm.

36. The lipstick of claim 18, wherein the lipstick further comprises one or more UV filters selected from 2,4,6-tris[anilino-(p-carbo-2′-ethyl-1′-hexyloxy)]-1,3,5-triazine (INCI: Ethylhexyl Triazone), 2-ethylhexyl 2-hydroxybenzoate (INCI: Ethylhexyl Salicylate), 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine), and 4-(tert-butyl)-4′-methoxydibenzoylmethane (INCI: Butyl Methoxydibenzoylmethane).

37. A method for producing the lipstick of claim 18, wherein the method comprises melting all constituents of the lipstick while stirring, casting the melted constituents into a casting mold at a temperature of from about 57° C. to 63° C. and removing the lipstick from the casting mold once the casting mold together with the cast lipstick has cooled at from 17° C. to 23° C. for a period of time.
